(19) United States
(12) Patent Application Publication
(10) Pub. No.: US 2025/0032769 A1
(43) Pub. Date: Jan. 30, 2025

(54) FLUID CONNECTOR ASSEMBLY

(71) Applicant: CareFusion 303, Inc., San Diego, CA (US)

(72) Inventors: Ryan CALLAHAN, Long Beach, CA (US); Seyed Alireza SALEHI BOROJERDI, Aliso Viejo, CA (US); Jason Andrew WINE, Brea, CA (US)

(21) Appl. No.: 18/360,717

(22) Filed: Jul. 27, 2023

(51) Int. Cl. A61M 39/10

(52) U.S. Cl. CPC A61M 39/10; A61M 2039/1061; A61M 2039/1077

(57) ABSTRACT

Fluid connector assemblies that seal off fluid paths in the respective connectors are disclosed. When connectors of a fluid connector assembly are connected to each other, respective compressible members in the connectors are displaced, allowing downstream fluid passage through the fluid connector assembly. The connectors maintain a connection by a snap mechanism that provides a threshold retention force. When an external force greater than the threshold force is applied to the fluid connector assembly, the snap mechanism may no longer maintain the connectors together, causing the connectors to decouple from each other. Once the connectors are decoupled, a connecting mechanism may be activated preventing the connectors to be coupled together without actuation of the connecting mechanism.

### TECHNICAL FIELD

The present disclosure relates generally to medical fluid connectors and, more particularly, to a fluid connector assembly that includes medical connectors that decouple with each other due to an applied force, with each medical connector designed to automatically seal off their respective fluid paths. The decoupling may be due to intentional or unintentional separation between the medical connectors.

### BACKGROUND

Peripheral intravenous (“PIVC”) catheters are medical tools inserted into peripheral veins of patients to deliver medical fluid to the patients. In an example application, the medical fluid is delivered to the patient, and a medical professional subsequently removes the PIVC catheter from the patient. Often, however, these catheters are unintentionally dislodged. For example, catheter lines receiving an unintended or unexpected pulling force can pull the IV tubing, which pulls the catheter out of the patient. In other instances, catheters are accidentally removed from patients and medical professionals. Unintended or unexpected dislodgement can lead to patient blood loss, IV fluid loss, and IV fluid delivery delay.

### SUMMARY

In accordance with at least some embodiments disclosed herein is the realization that unintended dislodgement or disconnection of a medical connection, such as a medical fluid line, can result in injury to a patient or a medical professional, such as by depriving the patient of a medicament, increasing the potential for infection to the patient, and exposing the medical professional to medicaments.

Aspects of the present disclosure provide fluid connector assemblies with medical connectors, each of which include one or more fluid paths, that respond to unintentional or unexpected external forces by decoupling from each other and sealing off their respective fluid paths. The decoupling may include automatic decoupling using bellows or other elastically compressible member that decompress and return to their original shape external forces are no longer acting upon them. Beneficially, fluid connector assemblies described herein can limit or prevent patient blood loss, IV fluid loss, infection, and medical delivery delays. Further, aspects of the present disclosure provide connecting mechanisms that aid in the connection of the fluid connector assembly in the event that the fluid connector assembly is decoupled.

According to certain embodiments, a fluid connector assembly includes a first connector having a first end and a second end, a second connector configured to couple to the first connector, and a connecting mechanism disposed on the first connector. The connecting mechanism may include a first end and a second end opposite the first end. The second end of the connecting mechanism may extend past the second end of the first connector. When the first connector and the second connector are disconnected, the connecting mechanism may remain disposed on the first connector.

In some embodiments, the connecting mechanism is formed of an elastomeric material. In some embodiments, the connecting mechanism is slidable along the first connector and includes a deployed position and a retracted position. In the deployed position, the second end of the connecting mechanism may extend beyond the second end of the first connector such that the second end of the first connector is positioned within an interior chamber of the connecting mechanism. In the retracted position, the second end of the connecting mechanism may be positioned closer to the first end of the connecting mechanism such that the second end of the first connector is positioned outside of the interior chamber. In in the retracted position, the second connector may be capable of being coupled to the first connector. In the deployed position, when the first connector and the second connector are coupled, the first connector and the second connector may be disposed within the interior chamber of the connecting mechanism. When a threshold force is applied to one of the first connector and the second connector, the first connector and second connector may be decoupled. When the first connector and the second connector are decoupled, the connecting mechanism may remain coupled to the first connector in the deployed position. When the first connector and second connector are decoupled and the connecting mechanism is in the deployed position, the connecting mechanism may prevent the first connector and the second connector from being coupled.

According to certain embodiments, a method for operating a fluid connector assembly includes providing a first connector, a second connector, and a connecting mechanism. The first connector may have a first end and a second end. The second connector may be configured to couple to the first connector. The connecting mechanism may be disposed on the first connector and include a first end and a second end opposite the first end. The method may further include positioning the connecting mechanism in a retracted position, connecting the first and second connectors, and positioning the connecting mechanism in a deployed position.

In some embodiments, positioning the connecting mechanism in the retracted position includes sliding the second end of the connecting mechanism along the first connector in a direction toward the first end such that the second end of the first connector is exposed.

In some embodiments, positioning the connecting mechanism in the deployed position includes sliding the connecting mechanism in a direction of the second end such that the connecting mechanism is disposed on both the first connector and the second connector.

In some embodiments, when a threshold force is applied to one of the first connector and the second connector, the first connector and the second connector may be decoupled. When the first connector and second connector or decoupled, the connecting mechanism may remain disposed on the first connector in the deployed position. The method may further include positioning the connecting mechanism in the retracted position such that an engaging surface of the first connector can be disinfected.

According to certain embodiments, a fluid connector assembly includes a first connector, a second connector, and a connecting mechanism. The first connector may include a first end and a second end opposite the first end. The second connector may include a first end and a second end opposite the first end and may be configured to be removably coupled to the first connector. The connecting mechanism may include a first end, a second end opposite the first end, and an interior chamber formed between the first end and the second end. The first connector may be disposed within the interior chamber. In a retracted position, the second end of the connecting mechanism may be positioned closer to the first end of the connecting mechanism such that the second end of the first connector is positioned outside of the interior chamber. In a deployed position, the second end of the connecting mechanism may extend beyond the second end of the first connector such that the second end of the first connector is positioned within the interior chamber of the connecting mechanism. When the connecting mechanism is in the retracted position, the second connector may be capable of being coupled to the first connector. When the first connector and the second connector are coupled, the connecting mechanism may be returned to the deployed position. When the first and second connector are decoupled, the connecting mechanism may remain in the deployed position.

In some embodiments, the connecting mechanism is slidable along the first connector between the retracted position and the deployed position.

In some embodiments, in the retracted position, the second end of the connecting mechanism may be positioned closer to the first end of the connecting mechanism such that the second end of the first connector is positioned outside of the interior chamber.

In some embodiments, when a threshold force is applied to one of the first connector and the second connector, the first connector and second connector may be decoupled. When the first connector and the second connector are decoupled, the connecting mechanism may remain coupled to the first connector in the deployed position.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a partial cross-sectional view of the connector of the fluid connector assembly, in accordance with aspects of the present disclosure.

FIG. 3 illustrates a partial cross-sectional view of the connector of the fluid connector assembly, in accordance with aspects of the present disclosure.

FIG. 4 illustrates a partial cross-sectional view of the fluid connector assembly, in accordance with aspects of the present disclosure.

FIG. 5 illustrates a partial cross-sectional view of the fluid connector assembly, in accordance with aspects of the present disclosure.

FIGS. 6A-6D illustrate views of the connecting mechanism of the fluid connector assembly, in accordance with aspects of the present disclosure.

FIG. 7 illustrates a perspective view of the fluid connector assembly with the connecting mechanism disposed on the first connector, in accordance with aspects of the present disclosure.

FIG. 8 illustrates a perspective view of the connecting mechanism of the fluid connector assembly in a retracted position, in accordance with aspects of the present disclosure.

FIG. 9 illustrates a perspective view of the fluid connector assembly and the connecting mechanism, in accordance with aspects of the present disclosure.

FIG. 10 illustrates a perspective view of the fluid connector assembly and the connecting mechanism, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of a fluid connector assembly with medical connectors that seal off their respective fluid paths when the medical connectors are decoupled from each other. The medical connectors may each include a compressible member that decompresses in response to the decoupling, thus providing an automatic sealing of the respective fluid paths. The fluid connector assembly may further include connecting mechanisms, which may function to prevent a patient from reconnecting the fluid connector assembly such that the respective surfaces may be properly disinfected.

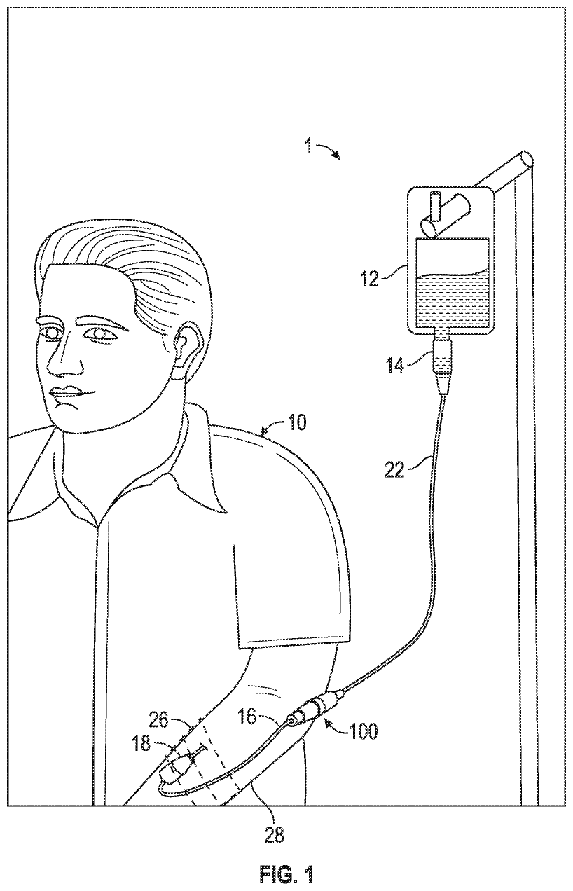

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector assembly 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

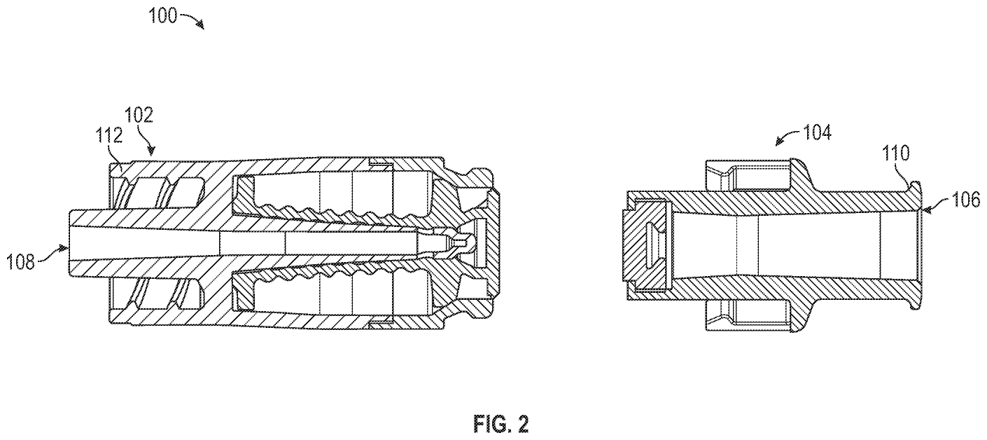

FIG. 2 illustrates a perspective view of a fluid connector assembly 100, in accordance with aspects of the present disclosure. The fluid connector assembly 100 is designed for use in medical applications, such as the IV set 1 (shown in FIG. 1) as well as other IV medical fluid delivery applications using catheters, including PIVC catheters, as non-limiting examples.

As shown, the fluid connector assembly 100 includes a connector 102 and a connector 104 coupled with the connector 102. The connector 102 and the connector 104 may be referred to as a first connector and a second connector, respectively. However, “first” and “second” may be interchangeable. Also, each of the connectors 102 and 104 may be referred to as medical connectors. When the connectors 102 and 104 are connected to each other, a fluid path for medical fluid is established by the fluid connector assembly 100.

In some embodiments, the connector 104 is connected to a medical fluid (not shown). Further, in some embodiments, the connector 102 is connected to a catheter line (not shown) that delivers the medical fluid to a catheter. In this regard, the connector 104 may include a fluid inlet 106 that acts as a fluid receiving location for the fluid connector assembly 100. Also, the connector 102 may include a fluid outlet 108 that acts as a fluid transmission location for the fluid connector assembly 100.

To facilitate the connection to the medical fluid, the connector 104 may include a luer 110. In some embodiments, the luer 110 is female luer designed to mate with a male connector that is connected to the medical fluid. Similarly, to facilitate the connection to the catheter line, the connector 102 may include a luer 112. In some embodiments, the luer 112 is male luer designed to mate with a female connector that is connected to the catheter line. Each of the luers 110, 112 may conform to standards established by the International Organization for Standards (“ISO”) to improve patient safety, minimize medical fluid leakage, and reduce misconnection with other connection devices.

Additionally, the connector 102 may include a post 128 passing centrally, or at least approximately centrally, through the connector 102. The post 128 may include a channel 130 that establishes the fluid outlet 108. While the post 128 is illustrated as cylindrical, the post 128 need not be so limited and may have any suitable shape.

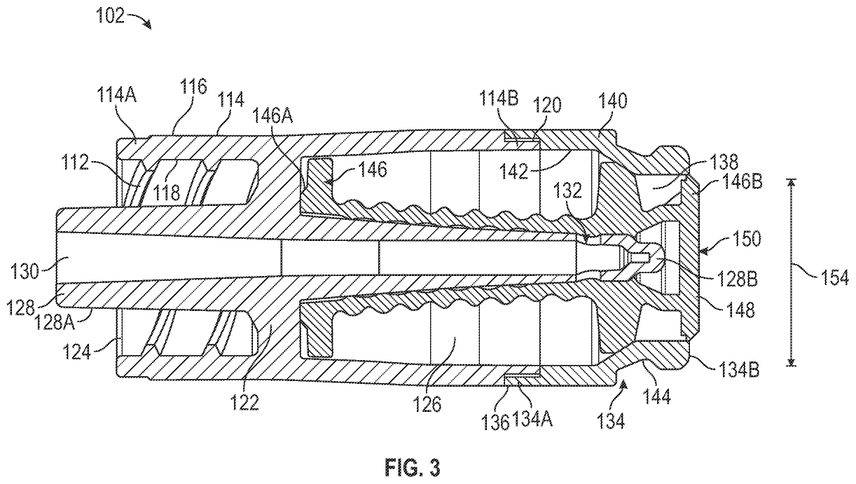

Referring to FIG. 3, the connector 102 may include a housing 114 integrated with the luer 112. The housing 114 may include a first end 114A having the integrated luer 112 and a second end 114B opposite the first end 114A configured to couple to a receiving ring 134. The second end 114B of the housing 114 may have a stepped profile with a protruding portion 120 having a reduced thickness relative the thickness of the rest of the housing. The second end 114B of the housing 114 may include an inner diameter 154.

The housing 114 may include an exterior surface 116 and an interior surface 118. The housing 114 may include a hollow, or generally hollow, body that carries one or more components. For example, in addition to the post 128, a compressible member 146, which will be discussed in greater detail, may be housed within the housing 114. The housing 114 may further include a wall 122 extending laterally across the interior of the connector 102 thereby separating the interior of the connector 102 into separate cavities 124, 126. The post 128 may be integrally formed with the wall 122 and extend through each of the cavities 124, 126. The post 128 may include an open first end 128A, a closed second end 128B opposite the first end 128A, and a channel 130 extending through the post 128. The first end 128A may include an opening for the channel 130, which forms the fluid outlet 108. The second end 128B of the post 128 may be a substantially closed end and include one or more apertures, also referred to as flow windows or openings, 132. The openings 132 may be fluidly connected to the channel 130 and the fluid outlet 108.

The connector 102 may further include the receiving ring 134 disposed at, and coupled to, the second end 114B of the housing 114. The receiving ring 134 may include a first end 134A configured to be coupled to the second end 114B and a second end 134B opposite the first end 134A. The first end 134A of the receiving ring 134 may have a stepped profile with a protruding portion 136 that complements the protruding portion 120 of the housing. The second end 134B of the receiving ring 134 may include an opening 138. The receiving ring 134 may have an exterior surface 140 and an interior surface 142. The receiving ring 134 may include one or more notches 144 formed along the exterior surface 140. In some embodiments, as shown in FIG. 3, the receiving ring 134 is coupled to the housing 114 of the connector 102. In other embodiments, the receiving ring 134 may be integrally formed with the housing 114 of the second connector 102 thereby forming a unitary construct.

As described above, the connector 102 may include a compressible member 146, also referred to a as a silicone valve, disposed within the housing 114. The compressible member 146 may be disposed within the cavity 126 of the housing 114 and extend through the receiving ring 134. The compressible member 146 may have a first end 146A positioned proximate a surface of the wall 122 and a second end 146B positioned within the opening 138 of the receiving ring 134 and configured to engage a component of the connector 104. The second end 146B may include an engagement surface 148. The engagement surface 148 may provide a planar, or generally planar, surface that can be readily accessed for cleaning and disinfecting. The engagement surface 148 may further include an opening 150. In some embodiments, the opening 150 may be formed as one or more slits or apertures. The post 128 may extend through the compressible member 146. In other words, the compressible member 146 may extend around an exterior surface of the post 128. The second end 128B of the post 128 may be positioned within an area formed proximate the second end 146B of the compressible member 146.

In some embodiments, the compressible member 146 includes a bellow that can elastically compress. Accordingly, the compressible member 146 can compress by an external force and subsequently return to its original, uncompressed form when the external force is removed. The compressible member 146 may be designed to regulate fluid flow through the connector 102.

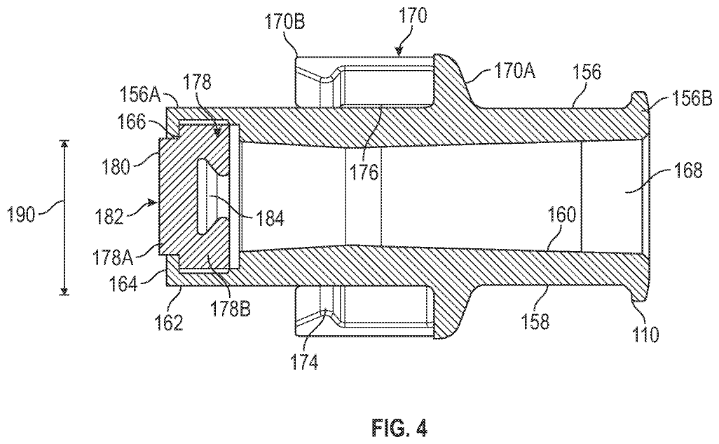

FIG. 4 illustrates a perspective view of the connector 104, showing additional features, in accordance with aspects of the present disclosure. The connector 104 may include a housing 156. The housing 114 (shown in FIG. 3) and the housing 156 may be referred to as a first housing and a second housing, respectively. However, “first” and “second” may be interchangeable. As shown, the housing 156 may be integrated with the luer 110.

The housing 156 may include a first end 156A configured to mate with the connector 102 when coupled and a second end 156B opposite the first end 156A. The housing 156 may include an exterior surface 158 and an interior surface 160. The housing 156 may include a channel 168 extending through the interior of the housing 156. The housing 156 may include a hollow, or generally hollow, body that carries one or more components. For example, the housing 156 may include a compressible member 178, located, or at least substantially located, in the housing 156.

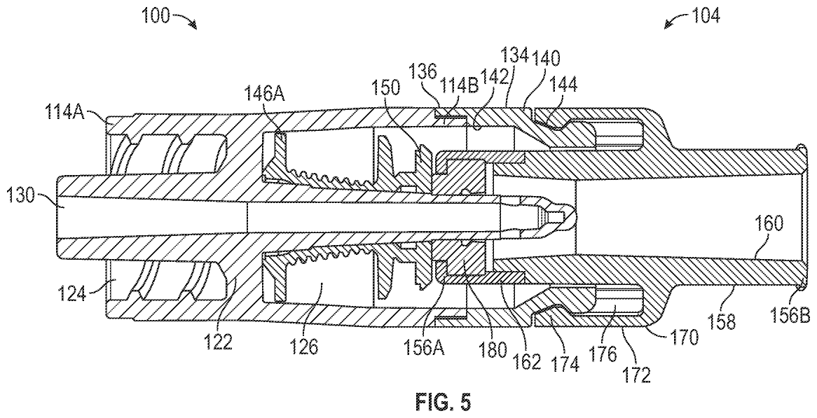

The outer diameter 190 may include a dimension that allows at least a portion of the compressible member 178 and at least a portion of the housing 156 to enter the connector 102 (shown in FIG. 5). In this manner, the dimension of the inner diameter 154 of the second end 114B (shown in FIG. 4) may be greater than the outer diameter 190 of the first end 156A of the housing 156 thereby allowing for the first end 156A of the connector 104 to be inserted within the second end 114B of the connector 102.

The connector 104 may include a forward portion 162 formed within the housing 156 at the first end 156A for receiving the compressible member 178. The housing 156 at the first end 156A may include an inwardly extending rim 164 thereby forming an end of the forward portion 162. The inwardly extending rim 164 may surround an opening 166 at the first end 156A of the housing 156. The exterior surface of the inwardly extending rim 164 may be designed to engage the engagement surface 148.

The connector 104 may further include a collar member 170 extending circumferentially around the exterior surface 158. The collar member 170 may include a first end 170A, a second end 170B disposed opposite the first end 170A, an exterior surface 170C, and interior surface 170D. The collar member 170 may further include one or more slits 172 extending longitudinally along the collar member 170. The slits 172 may allow for flexing of the collar member 170 when connecting the connectors 102, 104. The second connector 104 may further include a gap 174 formed between the exterior surface 158 of the connector 104 and the interior surface 170D of the collar member 170. A diameter of the collar member 170 may be sized so as to accommodate the second end 134B of the receiving ring 134.

As illustrated in FIG. 5, when coupling the connectors 102, 104, the second end 134B of the receiving ring 134 may be disposed within the gap 174 such that the interior surface 170D is disposed on an exterior surface 142 of the receiving ring 134. During the coupling the connectors 102, 104, the collar member 170 may flex outwardly via the slits 172 such that a size of the gap 174 increases thereby allowing the insertion of the receiving ring 134. Once the connectors 102, 104 are coupled, the collar 170 may return its original position such that the second end 170B is disposed within the notches 144 of the receiving ring 134 thereby forming a retaining force between the connectors 102, 104.

However, when an external force, such as a pulling force provided to one or more of the connectors 102 and 104, is applied to at least one of the connectors 102 and 104 and is greater than the threshold force, the collar member 170 may no longer maintain the connection/coupling between connectors 102 and 104, and the connectors 102 and 104 may decouple from each other. When this occurs, the forward portion 162 and the housing 156 of the connector 104 may be removed from the housing 114 of the connector 102.

The connector 104 may further include the compressible member, also referred to as an elastomeric plug, 178 disposed within the housing 156. In particular, the compressible member 178 may be disposed within the forward portion 162. The compressible member 178 may include a first end 178A positioned within the opening 166 and a second end 178B opposite the first end 178A. The first end 178A may include an engaging surface 180. The first end 178A may project out of the forward portion 162 of the housing 156 such that the engaging surface 180 is positioned forward of the inwardly extending rim 164. In other words, when the connectors 102, 104 are disconnected, the engaging surface 180 may be form an exterior surface of the housing 156. This positioning may allow the engaging surface 180 to be readily accessed for cleaning and disinfecting. The engaging surface 180 may include one or more apertures 182.

The compressible member 178 may further include an opening 184 formed at the second end 178B. While FIG. 4 shows a singular opening formed in the compressible member 178, the number of openings may vary. For example, in some embodiments, one or more openings may be formed in the compressible member 178. In either event, each opening(s) may include any feature shown and described for the openings 184. Fluid flowing through the fluid inlet 106 may pass through the compressible member 178 via the aperture 182. In some embodiments, the compressible member 178 includes a bellow that can elastically compress. Accordingly, the compressible member 178 can compress by an external force and subsequently return to its original, uncompressed form when the external force is removed. The compressible member 178 may be designed to regulate fluid flow through the connector 104.

Based on the connection, a seal may be formed between the housing 156 and the compressible member 178 such that fluid does not flow between the housing 156 and the compressible member 178 when the connector 104 is disconnected from the connector 102.

Based on the position of the compressible member 178 shown in FIG. 4, the connector 104 may prevent fluid flow therethrough. In other words, fluid that enters the channel 168 via the fluid inlet 106, may be prevented from passing through the forward portion 162 due to the positioning and expanded state of the compressible member 178.

In order to promote fluid through the connectors 102,104, when the fluid connector assembly 100 is assembled, the respective compressible members 146, 178 may be actuated, or displaced, to uncover respective openings in the connectors 102,104.

FIG. 5 illustrates a partial cross-sectional view of the fluid connector assembly 100, showing the connectors 102 and 104 engaged, and further showing actuation of the compressible members 146 and 178, in accordance with aspects of the present disclosure. The fluid connector assembly 100 can be assembled by coupling the connectors 102, 104. To couple the connectors, 102, 104, the respective engagement surfaces 148, 180 can be positioned such that they contact one another. Due to the respective dimensions of the inner diameter 154 of the connector 102 and the outer diameter 190 of the connector 104, the connector 104 may be received within the opening 138 of the receiving ring 134. Force, or pressure, may then be applied to either, or both of, the connectors 102, 104. As pressure is applied, the compressible members 146, 178 may begin to deform. For example, the compressible member 146 may deform such that the second end 146B of the compressible member 146 moves in a direction towards the first end 146A thereby positioning the second end 146B within the cavity of the receiving ring 134. As the compressible member 146 deforms under pressure, the second end 128B of the post 128 may protrude through the opening 150 of the engagement surface 148. The second end 146B of the compressible member 146 may continue to deform until the opening 132 of the post 128 are exposed thereby allowing fluids to enter the channel 130 of the post 128. Similarly, as pressure is applied to one or both of the connectors 102, 104, the first end 156A of the connector 104 may be moved within the receiving ring 134 and the compressible member 178 may deform such that the first end 178A of the compressible member 178 moves within the forward portion 162 toward the second end 178B. As the connector 104 is moved within the housing 114 of the connector 102, the second end 128B of the post 128 may contact the engagement surface 180 of the compressible member 178. In some embodiments, the post 128 may extend through the aperture 182 of the engagement surface 180.

The forward portion 162 of the connector 104 may be inserted into the receiving ring 134 and the housing 114 and can be at least partially inserted into the cavity 126 of the housing 114. The connectors 102, 104 may remain coupled together via the interconnection of the collar member 170 and the notch 144. In particular, the connectors 102, 104 can be held together via a friction or interference fit between collar member 170 and the notches 144.

Upon insertion of the housing 156, the forward portion 162 may engage the compressible member 146 of the connector 102. For example, as shown in FIG. 5, the compressible member 146 of the connector 102 may engage the compressible member 178 of the connector 104 such that the engaging surface 148 of the compressible member 146 contacts the engaging surface 180 of the compressible member 178. From the perspective of the compressible member 146, an external force may be provided by at least the forward portion 162 and/or engaging surface 180. Thus, based on the insertion and engagement described above, the engaging surface 180 and/or forward portion 162 may provide a force that actuates the compressible member 146.

For example, the actuation of the compressible member 146 may include a compression of the compressible member 146. In this manner, the compression of the compressible member 146 may cause the compressible member 146 to reduce in size, to move relative to the post 128 thereby exposing the opening 132 of the post 128. Accordingly, the opening 132 of the post 128 may be uncovered by the compressible member 146 and such that the compressible member 146 no longer provides a seal against fluid entry into the opening 132. As such, the compressible member 146, located in the housing 114, may be designed to actuate, e.g., compress, in response to a force received by an external object, or objects, of the connector 104.

In some embodiments, the post 128 of the connector 102 may engage the compressible member 146 of the connector 102 at or near the same time of insertion of the insertion of the forward portion 162 and the housing 156 into the housing 114. For example, the second end 128B of the post 128 may engage the engaging surface 180 of the compressible member 178 through the opening 150. From the perspective of the compressible member 178, an external force may be provided by at least the post 128. As the forward portion 162 and the housing 156 are further inserted into the housing 114, the post 128 may provide a force that actuates the compressible member 178. For example, the actuation of the compressible member 178 may include a compression of the compressible member 178, thereby reducing the size of the compressible member 178, forcing the first end 178A of the compressible member 178 out of the opening 166 of the forward portion thereby breaking the seal between the compressible member 178 and the first end 156A of connector 104. Also, the actuation of the compressible member 178 may allow the post 128 to pass through the opening 166 of the housing 156 and extend into an interior of the housing 156 at the forward portion 162. Moreover, the post 128 may pass through the opening 166 such that the opening 132 of the post 128 may also pass through the opening 166 and is positioned within an interior of the housing 156. Accordingly, the compressible member 178, located in the housing 156, may be designed to actuate, i.e., compress, in response to a force received by an external object, or objects, of the connector 102.

Based upon the configuration of the fluid connector assembly 100 a fluid path may be established. Accordingly, a fluid, including a medical fluid, can pass through the fluid connector assembly 100 downstream from the connector 104 to the connector 102. As shown, fluid can enter the fluid inlet 106 of the housing 156 and subsequently pass through the compressible member 178 by way of the opening 184 of the compressible member 178. The fluid can then pass through the opening 132 of the post 128, and then through the channel 130 of the post 128. The fluid can exit the fluid connector assembly 100 through the fluid outlet 108 of the housing 114.

As described above, the decoupling of the connectors 102 and 104 may result from an external force applied to one or more of the connectors 102 and 104, where the external force exceeds the threshold force provided by the collar member 170 that maintain a connection between the connectors 102 and 104.

When the connectors 102, 104 are decoupled, the force applied to the compressible members 146, 178 of the connectors 102, 104, respectively, may be removed such that the compressible members 146, 178 may decompress and expand such that the compressible members 146, 178 return to their original shapes. For example, during expansion of the compressible member 146, the second end 146B of the compressible member 146 may move toward the second end 134B of the receiving ring 134 until the compressible member 146 has returned to its original shape such of the and the second end 146B is positioned within the opening 138 of the receiving ring 134. Similarly, during expansion of the compressible member 178, the first end 178A may move toward the opening 166 until the compressible member 178 has returned to its original shape such that the first end 178A is positioned within the opening 166. As a result, fluid may be unable to flow upstream through the connector 102 and pass through the opening 132 of the post 128.

Each of the compressible members 146 and 178 may be used as a valve to regulate fluid through the fluid connector assembly 100. Based on their elastic, springlike properties, each of the compressible members 146 and 178 may automatically return to their respective shapes when external forces are no longer acting on them. Beneficially, the compressible members 146 and 178 may spring back in response to a decoupling between the connectors 102 and 104 to provide a relatively quick sealing effect. Accordingly, blood losses, IV fluid losses, and medical delivery delays can be limited or prevented, based on one or more properties of the compressible members 146 and 178, when integrating the fluid connector assembly 100 into an IV set and catheter.

In some embodiments, the fluid connector assembly 100 may further include a connecting mechanism 200 that activates in when the fluid connector assembly 100 is decoupled.

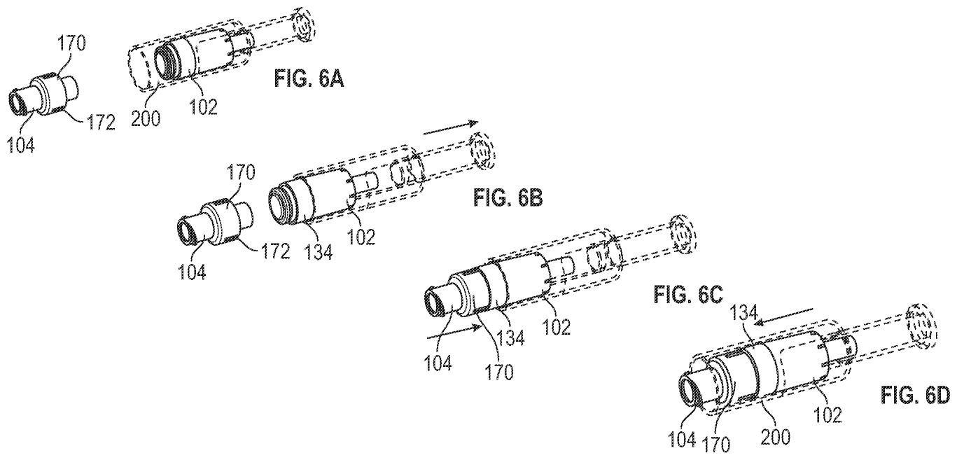

Referring to FIGS. 6-10, connecting mechanism 200, also referred to as a sleeve, may include a main body 202 having a first end 202A, a second end 202B opposite the first end, an exterior surface 202C, and an interior surface 202D. The interior surface 202D may form an interior chamber 204 within the main body 202 of the connecting mechanism 200. When assembled, the connector 102 may be disposed within the interior chamber 204 of the connecting mechanism 200 such that the interior surface 202D of main body 202 is disposed on the exterior surface 116 of the connector 102. Moreover, the main body 202 may be slidably coupled to the connector 102 such that the main body 202 is capable of sliding along a longitudinal axis of the connector 102. In some embodiments, the main body 202 further includes an exposed portion or window 206 thereby allowing a user to view the contents of the connector 102. Moreover, the exposed portion 206 may further allow for the deflection of the main body 202 during movement to the retracted portion.

In some embodiments, the connecting mechanism 200 is formed of an elastomeric material. However, the connecting mechanism 200 need not be so limited and may be formed of any suitable material capable of deformation and having elastomeric properties.

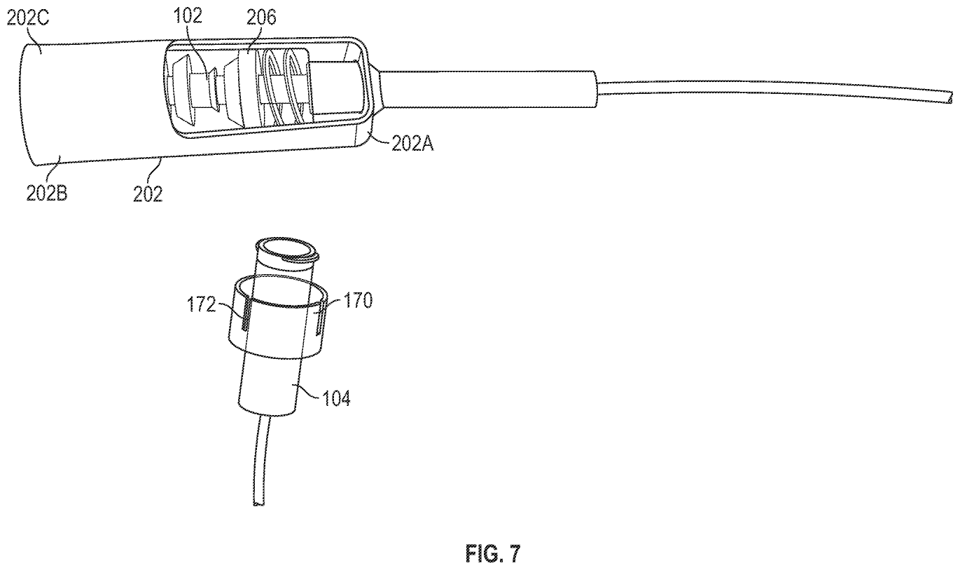

As shown in FIGS. 6-10, the connecting mechanism 200 may include a deployed position and a retracted position. As shown in FIG. 7, in the deployed position, the main body 202 may be disposed on the connector 102 such that the first end 202A of the main body 202 is positioned proximate the first end 114A of the connector 102 and the second end 202B of the main body 202 extends beyond the second end 114B and the receiving ring 134 of the connector 102 such that the second end 114B and the receiving ring 134 are disposed within the interior chamber 204. With the second end 114B of the connector 102 disposed within the interior chamber 204, the main body 202 may fully envelop the engagement surface 148 of the connector 102 thereby aiding in the prevention of unwanted contamination of the engagement surface 148 when the connectors 102, 104 are not coupled. Moreover, in the deployed position, a user may be prevented from coupling the connectors 102, 104 until actuation of the connecting mechanism 200.

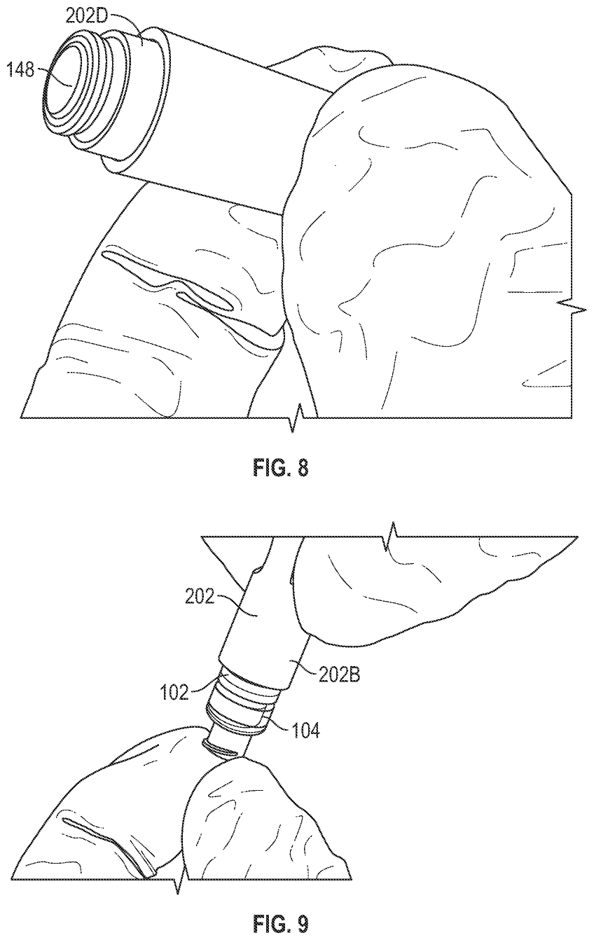

Referring to FIG. 8, in the retracted position, the second end 202B of the main body 202 may be positioned closer to the first end 202A of the main body 202 such that the second end 114B and/or receiving ring 134 of the connector 102 is positioned outside of the interior chamber 204. The retracted position may therefore allow the engagement surface 148 to be exposed. When the connecting mechanism 200 is in the retracted position, a user may be able to couple the connectors 102, 104.

FIGS. 6A-6D illustrate the operation of the connecting mechanism 200. In some embodiments, the connecting mechanism 200 may be actuated by a user to move between the deployed and retracted positions. For example, a user may grip portion of the main body 202 and slide the main body 202 relative the connector 102 in a direction of the first end 114A of the connector 102. Consequently, the second end 202B of the main body 202 may be moved in a direction of the first end 202A of the main body 202 thereby exposing the second end 114B and/or receiving ring 134 of the connector 102. With the connecting mechanism 200 in the retracted position, a user may couple the connectors 102, 104 via the process described above, such that the connectors 102, 104 are connected as illustrated in FIG. 9.

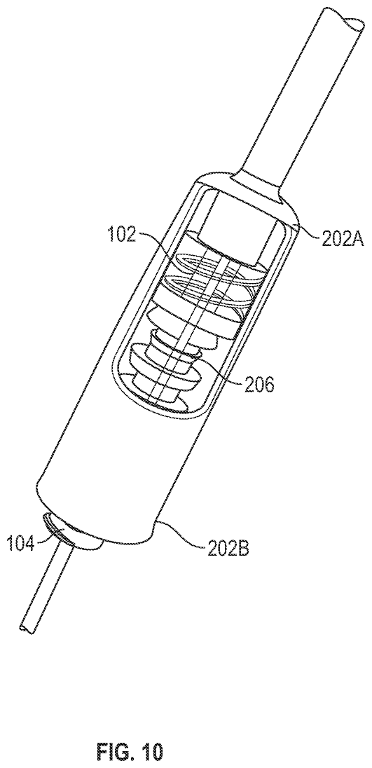

A user may then actuate the connecting mechanism 200 to return the connecting mechanism 200 to the deployed position, as shown in FIG. 10. For example, a user may grip the main body 202 and slide the main body 202 toward the second end 114B of the connector 202 such that the main body 202 slides longitudinally along the connector 102. The main body 202 may continue to slide in the direction of the second end 114B until the second end 114B of the connector 102, as well as the connector 104, are positioned within the interior chamber 204. With the connectors 102, 104 coupled and the connecting mechanism 200 in the deployed position, the connecting mechanism 200 may further aid in preventing unwanted disconnection of the connectors 102, 104.

As described above, when an external force that exceeds the threshold force is applied to one or more of the connectors 102, 104, the connectors 102, 104 may be decoupled. In the event that the connectors 102, 104 are decoupled, the connecting mechanism 200 may remain in the deployed position and disposed on the connector 102. As such, after the connectors 102, 104 are decoupled, the second end 114B and the engaging surface 148 may remain disposed within the interior chamber 204 of the connecting mechanism 200. In other words, during a disconnection event, the connecting mechanism 200 may aid in isolating the connectors 102, 104, to prevent reconnection prior to disinfection of the engaging surface 148. Once disconnected, a user may then actuate the connecting mechanism 200 to the retracted position to allow for disinfection of the engaging surface 148 before reconnecting the connectors 102, 104.

The features of the present disclosure provide first and second compressible members that can be used as valves to regulate a fluid pathway therebetween. The first and second compressible members are located in a first and a second connector, respectively. If the first and second connectors are separated, whether unintentionally or intentionally, the fluid pathway for each of the first and second compressible members become closed or obstructed to prevent fluid loss therefrom. The features of the present disclosure also provide that upon separation of the first and second compressible members, any of the first and second compressible members can be cleaned and disinfected, and the first and second compressible members can be once again coupled together to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 11, clause 17, clause, clause 23, clause 26, or clause 29. The other clauses can be presented in a similar manner.

Clause 1. A fluid connector assembly comprising: a first connector having a first end and a second end; a second connector configured to couple to the first connector; and a connecting mechanism disposed on the first connector, wherein the connecting mechanism comprises a first end and a second end opposite the first end, wherein the second end of the connecting mechanism extends past the second end of the first connector, and wherein, when the first connector and the second connector are disconnected, the connecting mechanism remains disposed on the first connector.

Clause 2. The fluid connector assembly described above, wherein the connecting mechanism is formed of an elastomeric material.

Clause 3. The fluid connector assembly described above, wherein the connecting mechanism is slidable along the first connector and comprises a deployed position and a retracted position.

Clause 4. The fluid connector assembly described above, wherein, in the deployed position, the second end of the connecting mechanism extends beyond the second end of the first connector such that the second end of the first connector is positioned within an interior chamber of the connecting mechanism, and wherein, in the retracted position, the second end of the connecting mechanism is positioned closer to the first end of the connecting mechanism such that the second end of the first connector is positioned outside of the interior chamber.

Clause 5. The fluid connector assembly described above, wherein, in the retracted position, the second connector is capable of being coupled to the first connector, and wherein, in the deployed position, when the first connector and the second connector are coupled, the first connector and the second connector are disposed within the interior chamber of the connecting mechanism.

Clause 6. The fluid connector assembly described above, wherein, when a threshold force is applied to one of the first connector and the second connector, the first connector and second connector are decoupled.

Clause 7. The fluid connector assembly described above wherein, when the first connector and the second connector are decoupled, the connecting mechanism remains coupled to the first connector in the deployed position.

Clause 8. The fluid connector assembly described above, wherein, when the first connector and second connector are decoupled and the connecting mechanism is in the deployed position, the connecting mechanism prevents the first connector and the second connector from being coupled.

Clause 9. A method for operating a fluid connector assembly comprising: providing a first connector having a first end and a second end, providing a second connector configured to couple to the first connector, providing a connecting mechanism disposed on the first connector, wherein the connecting mechanism comprises a first end and a second end opposite the first end, positioning the connecting mechanism in a retracted position, connecting the first and second connectors, and positioning the connecting mechanism in a deployed position.

Clause 10. The method, wherein the connecting mechanism is formed of an elastomeric material.

Clause 11. The method described above, wherein positioning the connecting mechanism in the retracted position comprises sliding the second end of the connecting mechanism along the first connector in a direction toward the first end such that the second end of the first connector is exposed.

Clause 12. The method described above wherein positioning the connecting mechanism in the deployed position comprises sliding the connecting mechanism in a direction of the second end such that the connecting mechanism is disposed on both the first connector and the second connector.

Clause 13. The method described above wherein, when a threshold force is applied to one of the first connector and the second connector, the first connector and the second connector are decoupled, and wherein, when the first connector and second connector or decoupled, the connecting mechanism remains disposed on the first connector in the deployed position.

Clause 14. The method described above further comprising the step of positioning the connecting mechanism in the retracted position such that an engaging surface of the first connector can be disinfected.

Clause 15. A fluid connector assembly comprising: a first connector having a first end and a second end opposite the first end; a second connector having a first end and a second end opposite the first end, the second connector configured to be removably coupled to the first connector; a connecting mechanism comprising: a first end, a second end opposite the first end, and an interior chamber formed between the first end and the second end, wherein the first connector is disposed within the interior chamber, wherein, in a retracted position, the second end of the connecting mechanism is positioned closer to the first end of the connecting mechanism such that the second end of the first connector is positioned outside of the interior chamber; wherein, in a deployed position, the second end of the connecting mechanism extends beyond the second end of the first connector such that the second end of the first connector is positioned within the interior chamber of the connecting mechanism, wherein, when the connecting mechanism is in the retracted position, the second connector is capable of being coupled to the first connector, wherein, when the first connector and the second connector are coupled, the connecting mechanism is returned to the deployed position, and wherein, when the first and second connector are decoupled, the connecting mechanism remains in the deployed position.

Clause 16. The fluid connector assembly described above, wherein the connecting mechanism is formed of an elastomeric material.

Clause 17. The fluid connector assembly described above, wherein the connecting mechanism is slidable along the first connector between the retracted position and the deployed position.

Clause 18. The fluid connector assembly described above, wherein, in the retracted position, the second end of the connecting mechanism is positioned closer to the first end of the connecting mechanism such that the second end of the first connector is positioned outside of the interior chamber.

Clause 19. The fluid connector assembly described above, wherein, when a threshold force is applied to one of the first connector and the second connector, the first connector and second connector are decoupled.

Clause 20. The fluid connector assembly described above wherein, when the first connector and the second connector are decoupled, the connecting mechanism remains coupled to the first connector in the deployed position.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector assembly comprising:
a first connector having a first end and a second end;
a second connector configured to couple to the first connector; and
a connecting mechanism disposed on the first connector, wherein the connecting mechanism comprises a first end and a second end opposite the first end,
wherein the second end of the connecting mechanism extends past the second end of the first connector, and
wherein when the first connector and the second connector are disconnected, the connecting mechanism remains disposed on the first connector.

2. The fluid connector assembly of claim 1, wherein the connecting mechanism is formed of an elastomeric material.

3. The fluid connector assembly of claim 1, wherein the connecting mechanism is slidable along the first connector and comprises a deployed position and a retracted position.

4. The fluid connector assembly of claim 3, wherein in the deployed position, the second end of the connecting mechanism extends beyond the second end of the first connector such that the second end of the first connector is positioned within an interior chamber of the connecting mechanism, and wherein, in the retracted position, the second end of the connecting mechanism is positioned closer to the first end of the connecting mechanism such that the second end of the first connector is positioned outside of the interior chamber.

5. The fluid connector assembly of claim 4, wherein in the retracted position, the second connector is capable of being coupled to the first connector, and wherein, in the deployed position, when the first connector and the second connector are coupled, the first connector and the second connector are disposed within the interior chamber of the connecting mechanism.

6. The fluid connector assembly of claim 5, wherein when a threshold force is applied to one of the first connector and the second connector, the first connector and second connector are decoupled.

7. The fluid connector assembly of claim 6, wherein when the first connector and the second connector are decoupled, the connecting mechanism remains coupled to the first connector in the deployed position.

8. The fluid connector assembly of claim 7, wherein when the first connector and second connector are decoupled and the connecting mechanism is in the deployed position, the connecting mechanism prevents the first connector and the second connector from being coupled.

9. A method for operating a fluid connector assembly comprising:
providing a first connector having a first end and a second end,
providing a second connector configured to couple to the first connector,
providing a connecting mechanism disposed on the first connector, wherein the connecting mechanism comprises a first end and a second end opposite the first end,
positioning the connecting mechanism in a retracted position,
connecting the first and second connectors, and
positioning the connecting mechanism in a deployed position.

10. The method of claim 9, wherein the connecting mechanism is formed of an elastomeric material.

11. The method of claim 9, wherein positioning the connecting mechanism in the retracted position comprises sliding the second end of the connecting mechanism along the first connector in a direction toward the first end such that the second end of the first connector is exposed.

12. The method of claim 9, wherein positioning the connecting mechanism in the deployed position comprises sliding the connecting mechanism in a direction of the second end such that the connecting mechanism is disposed on both the first connector and the second connector.

13. The method of claim 9, wherein when a threshold force is applied to one of the first connector and the second connector, the first connector and the second connector are decoupled, and
wherein, when the first connector and second connector or decoupled, the connecting mechanism remains disposed on the first connector in the deployed position.

14. The method of claim 13, further comprising the step of positioning the connecting mechanism in the retracted position such that an engaging surface of the first connector can be disinfected.

15. A fluid connector assembly comprising:
a first connector having a first end and a second end opposite the first end;
a second connector having a first end and a second end opposite the first end, the second connector configured to be removably coupled to the first connector;
a connecting mechanism comprising:
a first end,
a second end opposite the first end, and
an interior chamber formed between the first end and the second end,
wherein the first connector is disposed within the interior chamber,
wherein, in a retracted position, the second end of the connecting mechanism is positioned closer to the first end of the connecting mechanism such that the second end of the first connector is positioned outside of the interior chamber;
wherein, in a deployed position, the second end of the connecting mechanism extends beyond the second end of the first connector such that the second end of the first connector is positioned within the interior chamber of the connecting mechanism,
wherein, when the connecting mechanism is in the retracted position, the second connector is capable of being coupled to the first connector,
wherein, when the first connector and the second connector are coupled, the connecting mechanism is returned to the deployed position, and
wherein, when the first and second connector are decoupled, the connecting mechanism remains in the deployed position.

16. The fluid connector assembly of claim 15, wherein the connecting mechanism is formed of an elastomeric material.

17. The fluid connector assembly of claim 15, wherein the connecting mechanism is slidable along the first connector between the retracted position and the deployed position.

18. The fluid connector assembly of claim 15, wherein in the retracted position, the second end of the connecting mechanism is positioned closer to the first end of the connecting mechanism such that the second end of the first connector is positioned outside of the interior chamber.

19. The fluid connector assembly of claim 15, wherein when a threshold force is applied to one of the first connector and the second connector, the first connector and second connector are decoupled.

20. The fluid connector assembly of claim 19, wherein when the first connector and the second connector are decoupled, the connecting mechanism remains coupled to the first connector in the deployed position.
